(19) United States
(12) Patent Application Publication
(10) Pub. No.: US 2018/0103963 A1
(43) Pub. Date: Apr. 19, 2018

(54) MEDICAL DEVICE HANDPIECE WITH INTERCHANGEABLE ROTARY HEAD AND DRILL BIT

(71) Applicant: Arthrex, Inc., Naples, FL (US)

(72) Inventors: James P. Bradley, Pittsburgh, PA (US); William R. Beach, Glen Allen, VA (US); Philip S. O'Quinn, Naples, FL (US); Stephen Michael Cottle, Naples, FL (US)

(73) Assignee: Arthrex, Inc., Naples, FL (US)

(21) Appl. No.: 15/292,784

(22) Filed: Oct. 13, 2016

(51) Int. Cl. A61B 17/16

(52) U.S. Cl. CPC A61B 17/1617; A61B 2017/00464; A61B 17/1622; A61B 17/1633

(57) ABSTRACT

A rotary medical device configured to be used to prepare a surgical site within a patient for affixing at least one fixation device via a rotary abrading element and a rotary drill bit, both of which are attachable to the same handpiece, is disclosed. By configuring the rotary medical device to operate both a rotary abrading element and a rotary drill bit, the need for a separate, single use drill and the time and human resources needed to disinfect and process the drill is eliminated, thereby saving time and cost. In at least one embodiment, the rotary medical device may include a first detachable system including a first abrading element attached to a first detachable hub, which is attachable to the handpiece, and a second detachable system including a first drill bit attached to a second detachable hub, which is also attachable to the handpiece.

### FIELD OF THE INVENTION

The invention relates to rotary abraders useful in arthroscopy, and more particularly, to rotary abraders for abrading tissue and drills for drilling holes in tissue.

### BACKGROUND

During arthroscopic surgery, orthopedists perform procedures using special purpose instruments designed specifically for arthroscopic surgery. Among these special purpose tools are shaver blades, rotary abraders and drills. Shaver blades having hollow bores are typically removably coupled to a shaver handpiece and are used for cutting, resecting, boring and abrading both soft and hard tissue at the surgical site. An arthroscopic abrader generally includes a rotatable inner tube having an abrading head at its distal end and a fixed outer tube for rotatably receiving the inner tube. Abraders are used for abrading or shaping both soft and hard tissue such as bone, cartilage, ligaments, etc. by use of the rotating abrading head. As the tissue is being abraded, debris and fluid are generally drawn or sucked through the rotatable inner tube. Conventional drills are used to drill holes in tissue at surgical sites to facilitate attachment of fixation devices to the patient. In order to conduct a surgical procedure, all tools must be processed to sterilize the tools before surgery. Such processing is costly and utilizes human resources. Thus, a need exists for a more efficient surgical tool system.

### SUMMARY OF THE INVENTION

A rotary medical device configured to be used to prepare a surgical site within a patient for affixing at least one fixation device via a rotary abrading element and a rotary drill bit, both of which are attachable to the same handpiece, is disclosed. By configuring the rotary medical device to operate both a rotary abrading element and a rotary drill bit, the need for a separate single use drill and the time and human resources needed to disinfect and process the drill is eliminated, thereby saving time and cost. In at least one embodiment, the rotary medical device may include a first detachable system including a first abrading element attached to a first detachable hub, which is attachable to the handpiece, and a second detachable system including a first drill bit attached to a second detachable hub, which is also attachable to the handpiece. As such, a surgeon may use the handpiece to both abrade tissue at surgical site within a patient and drill a hole within tissue at the surgical site.

In at least one embodiment, the medical device may be formed from an interchangeable head for abrading tissue that is formed from a handpiece, an inner drive shaft rotatably positioned within the handpiece, a receiver hub formed at a distal end of the handpiece and the inner drive shaft and a first detachable system. The first detachable system may be formed from an outer housing, an inner drive shaft rotatably positioned within the outer housing, a first abrading element attached to a distal end of the inner drive shaft and at least partially extending from the outer housing, whereby the first abrading element is configured to be used to abrade tissue. The first detachable system may also include a first detachable hub attached to the outer housing and configured to couple the first detachable hub to the receiver hub such that the inner drive shaft of the first detachable system is placed in mechanical communication with the inner drive shaft positioned within the handpiece. The medical device may include a second detachable system including a first drill bit extending distally from a second detachable hub, whereby the first drill bit is configured to be used to drill a hole in tissue. The second detachable hub may be configured to couple the second detachable hub to the receiver hub such that the first drill bit of the second detachable system is placed in mechanical communication with the inner drive shaft positioned within the handpiece.

In at least one embodiment, the first abrading element is a burr. In another embodiment, the first abrading element is a shaver. The first drill bit may be formed from an elongated shaft with at least one flute extending proximally from a cutting edge at a distal end of the elongated shaft. The first drill bit may have a cutting edge configured to create a hole with a first diameter.

The medical device may also include a third detachable system formed from a second drill bit extending distally from a third detachable hub, whereby the second drill bit is configured to be used to drill a hole in tissue, and the hole has a second diameter that differs from the first diameter of a hole formed by the first drill bit. The third detachable hub may be configured to couple the third detachable hub to the receiver hub such that the second drill bit of the third detachable system is placed in mechanical communication with the inner drive shaft positioned within the handpiece.

A method of preparing a surgical site within a patient for affixing at least one fixation device may include abrading tissue at the surgical site with a medical device. Abrading tissue at the surgical site with a medical device may include using the medical device formed from a handpiece, an inner drive shaft rotatably positioned within the handpiece, a receiver hub formed at a distal end of the handpiece and the inner drive shaft and a first detachable system. The first detachable system may be formed from an outer housing, an inner drive shaft rotatably positioned within the outer housing, a first abrading element attached to a distal end of the inner drive shaft and at least partially extending from the outer housing, whereby the first abrading element is configured to be used to abrade tissue, and a first detachable hub attached to the outer housing and coupled to the first detachable hub to the receiver hub such that the inner drive shaft of the first detachable system is placed in mechanical communication with the inner drive shaft positioned within the handpiece. The medical device may also be formed from a second detachable system including a first drill bit extending distally from the outer housing, wherein the first drill bit is configured to be used to drill a hole in tissue, and a second detachable hub configured to couple the second detachable hub to the receiver hub such that of the second detachable system is placed in mechanical communication with the inner drive shaft positioned within the handpiece. The step of abrading tissue at the surgical site with a medical device may include abrading tissue at the surgical site with a medical device with a first detachable system having the first abrading element configured as a burr.

The method of preparing a surgical site within a patient may also include drilling a hole in tissue at the surgical site with the medical device, whereby the second detachable system is attached to the handpiece such that the second detachable hub is coupled with the receiver hub on the handpiece and the first detachable system is detached from the receiver hub on the handpiece. The step of drilling the hole in tissue at the surgical site with the medical device may include drilling the hole in tissue at the surgical site with the medical device with the second detachable system including the first drill bit formed from an elongated shaft with at least one flute extending proximally from a cutting edge at a distal end of the elongated shaft. The step of drilling the hole in tissue at the surgical site with the medical device may include drilling the hole in tissue at the surgical site with the medical device with the second detachable system including the first drill bit having a cutting edge configured to create a hole with a first diameter.

The step of drilling the hole in tissue at the surgical site with the medical device may include drilling the hole in tissue at the surgical site with the medical device with a third detachable system. The third detachable system may include a second drill bit extending distally from a third detachable hub, whereby the second drill bit is configured to be used to drill a hole in tissue, and the hole has a second diameter that differs from the first diameter of a hole formed by the first drill bit. The step of drilling the hole in tissue at the surgical site with the medical device may include drilling the hole in tissue at the surgical site with the medical device with either the second detachable system having the first drill bit configured to create a hole or the third detachable system having the second drill bit configured to create a hole with a diameter different than a diameter of a hole created by the first drill bit.

An advantage of the invention is that a user, such as, but not limited to, a surgeon, may use the handpiece to both abrade tissue at surgical site within a patient and drill a hole within tissue at the surgical site.

Another advantage of the invention is that by configuring the rotary medical device to operate both a rotary abrading element and a rotary drill bit, the need for a separate single use drill and the time and human resources needed to disinfect and process the drill is eliminated, thereby saving time and cost.

These and other embodiments are described in more detail below.

### BRIEF DESCRIPTION OF THE FIGURES

FIG. 1 is a perspective view of the rotary medical device with first, second and third detachable systems formed from a rotary abrading element, a first drill bit and a second drill bit respectively.

FIG. 2 is a perspective view of surgical site with holes drilled in tissue with the first and second drill bits.

FIG. 3 is a perspective view of the first detachable system.

FIG. 4 is a perspective view of a drill bit.

FIG. 5 is a perspective view of the second detachable system.

FIG. 6 is a flow diagram of a method of using the rotary medical device.

FIG. 7 is a partial perspective view of a shaver usable with the rotary medical device.

### DETAILED DESCRIPTION OF THE FIGURES

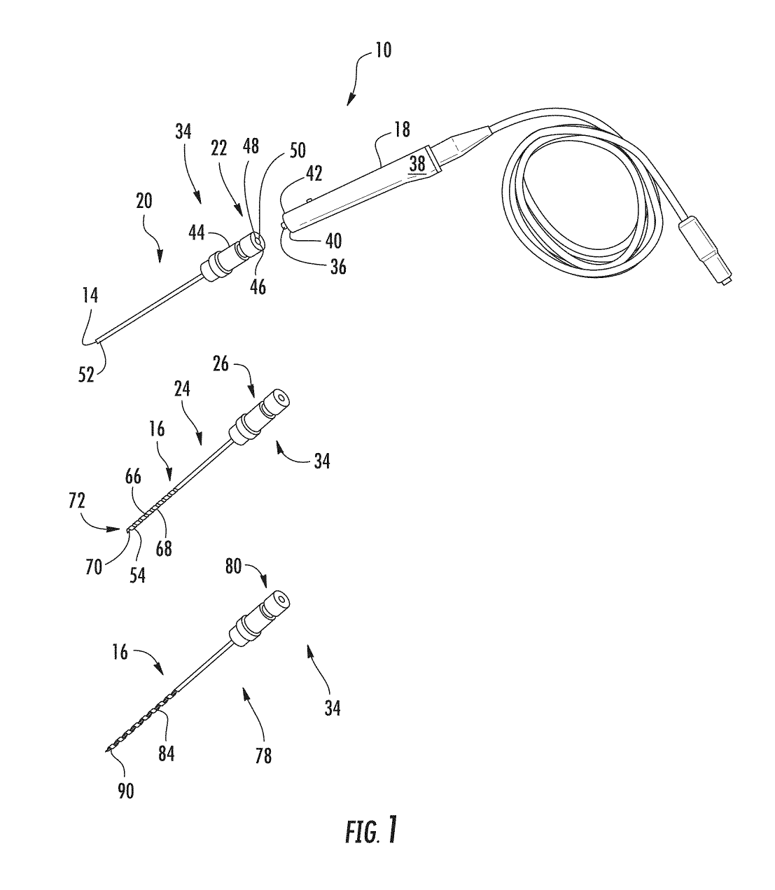

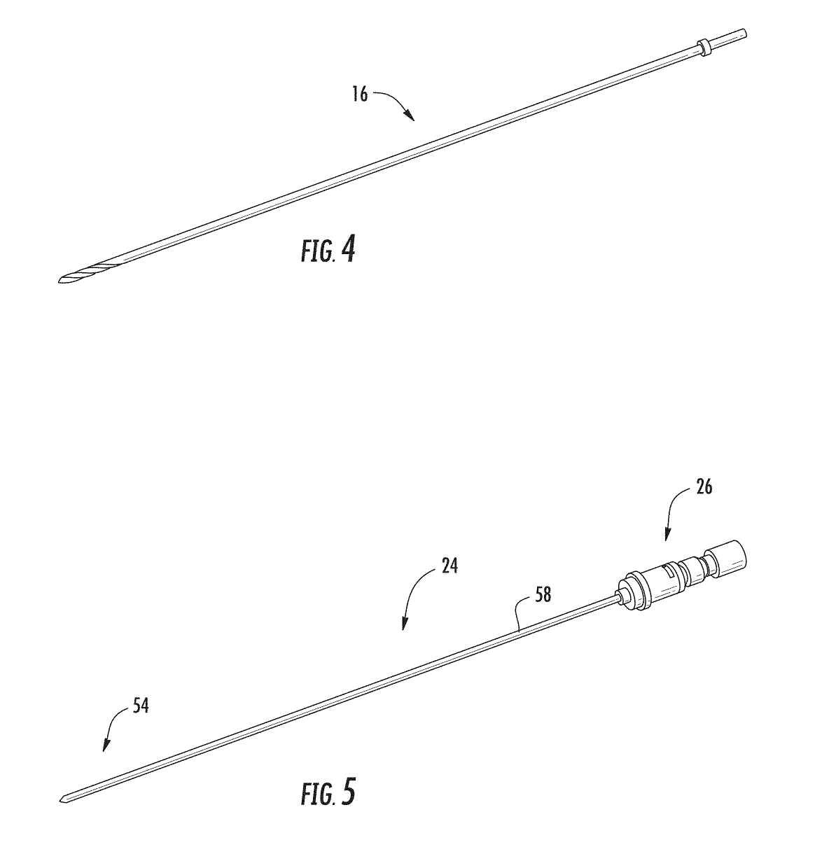

As shown in FIGS. 1-7, a rotary medical device 10 configured to be used to prepare a surgical site 30 within a patient for affixing at least one fixation device 12 via a rotary abrading element 14 and a rotary drill bit 16, both of which are attachable to the same handpiece 18, is disclosed. By configuring the rotary medical device 10 to operate both a rotary abrading element 14 and a rotary drill bit 16, the need for a separate single use drill and the time and human resources needed to disinfect and process the drill is eliminated, thereby saving time and cost. In at least one embodiment, the rotary medical device 10 may include a first detachable system 20 including a first abrading element 14 attached to a first detachable hub 22, which is attachable to the handpiece 18, and a second detachable system 24 including a first drill bit 54 attached to a second detachable hub 26, which is also attachable to the handpiece 18. As such, a surgeon may use the handpiece 18 to both abrade tissue 28 at surgical site 30 within a patient and drill a hole 32 within tissue 28 at the surgical site 30.

In at least one embodiment, the medical device 10 may be formed from an interchangeable head 34 for manipulating tissue 28, such as abrading tissue 28. The medical device 10 may include one or more handpieces 18 and an inner drive shaft 36 rotatably positioned within the handpiece 18. The handpiece 18 may have any appropriate shape and configuration, and in at least one embodiment, may have an outer surface 38 ergonomically configured to fit within a hand of a user, such as, but not limited to, a surgeon. As such, the handpiece 18 may be a handheld unit. The inner drive shaft 36 may be rotatably positioned within the handpiece 18 and may be driven via a motor (not shown) within the handpiece 18 or positioned elsewhere. The inner drive shaft 36 may be formed from any appropriate material, such as, but not limited to, stainless steel.

The medical device 10 may include a receiver hub 40 formed at a distal end 42 of the handpiece 18 and the inner drive shaft 36. The medical device 10 may include a first detachable system 20 with a first abrading element 14. The first detachable system 20 may include an outer housing 44 and an inner drive shaft 46 rotatably positioned within the outer housing 44. The outer housing 44 and an inner drive shaft 46 may have any appropriate configuration. In at least one embodiment, the outer housing 44 may have a cylindrical chamber 48 extending therethrough that has a larger diameter than an outer surface 50 of the inner drive shaft 46. One or more first abrading elements 14 may be attached to a distal end 52 of the inner drive shaft 46 and may at least partially extend from the outer housing 44. The first abrading element 14 may be configured to be used to abrade tissue. In at least one embodiment, the first abrading element 14 may be a burr 14. In at least one embodiment, the first abrading element 14 may be a shaver 15, as shown in FIG. 7.

The first detachable system 20 may include a first detachable hub 22 attached to the outer housing 44 and configured to couple the first detachable hub 22 to the receiver hub 40 such that the inner drive shaft 46 of the first detachable system 20 is placed in mechanical communication with the inner drive shaft 46 positioned within the handpiece 18. The first detachable hub 22 may have any appropriate configuration enabling the first detachable hub 22 and attached components to be removably attached to the receiver hub 40 on the handpiece 18.

The medical device 10 may include a second detachable system 24 configured to support the first drill bit 54. The first drill bit 54 may be configured to be used to drill a hole 32 in tissue 28. The first drill bit 54 may be formed from an elongated shaft 66 with at least one flute 68 extending proximally from a cutting edge 70 at a distal end 72 of the elongated shaft 66. The cutting edge 70 of the first drill bit 54 may be configured to create a hole 32 with a first diameter 76. In at least one embodiment, the first drill bit 54 may be formed from a step drill bit with a tip portion having a smaller diameter than a base portion.

The second detachable system 24 may also include a second detachable hub 26 configured to couple the second detachable hub 26 to the receiver hub 40 such that the first drill bit 54 of the second detachable system 24 is placed in mechanical communication with the inner drive shaft 36 positioned within the handpiece 18. The second detachable hub 26 may have any appropriate configuration enabling the second detachable hub 26 and attached components to be removably attached to the receiver hub 40 on the handpiece 18. The first drill bit 54 may be extend distally from the second detachable hub 26. The first drill bit 54 may be attached directly to the second detachable hub 26 or may be attached indirectly to the second detachable hub 26.

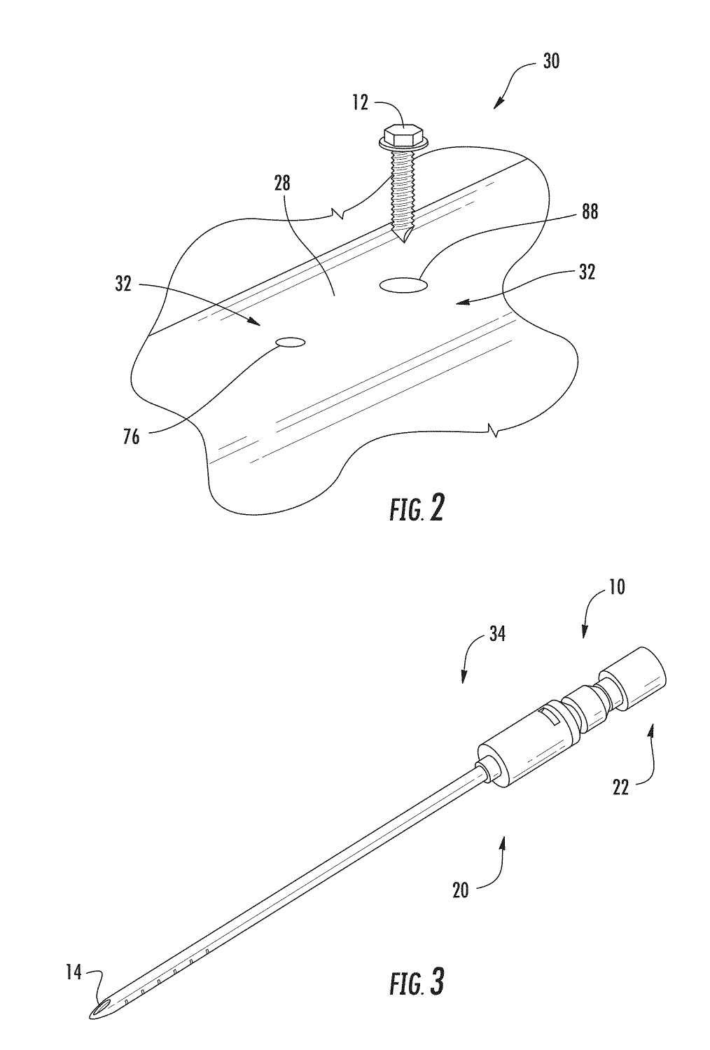

In at least one embodiment, the medical device 10 may include a third detachable system 78 including a second drill bit 84 configured to be used to drill a hole 32 in tissue 28, whereby the hole 32 has a second diameter 88 that differs from the first diameter of a hole 32 formed by the first drill bit 54, as shown in FIG. 2. As such, an outer diameter of the second drill bit 84 at a cutting edge 90 of the second drill bit 84 differs from an outer diameter of the first drill bit 54 at the cutting edge 70 of the first drill bit 54. The third detachable system 78 may include a third detachable hub 80 configured to couple the third detachable hub 80 to the receiver hub 40 such that the second drill bit 84 of the third detachable system 80 is placed in mechanical communication with the inner drive shaft 36 positioned within the handpiece 18. The second drill bit 84 may extend distally from the third detachable hub 80. The second drill bit 84 may be attached directly to the third detachable hub 80 or may be attached indirectly to the third detachable hub 80.

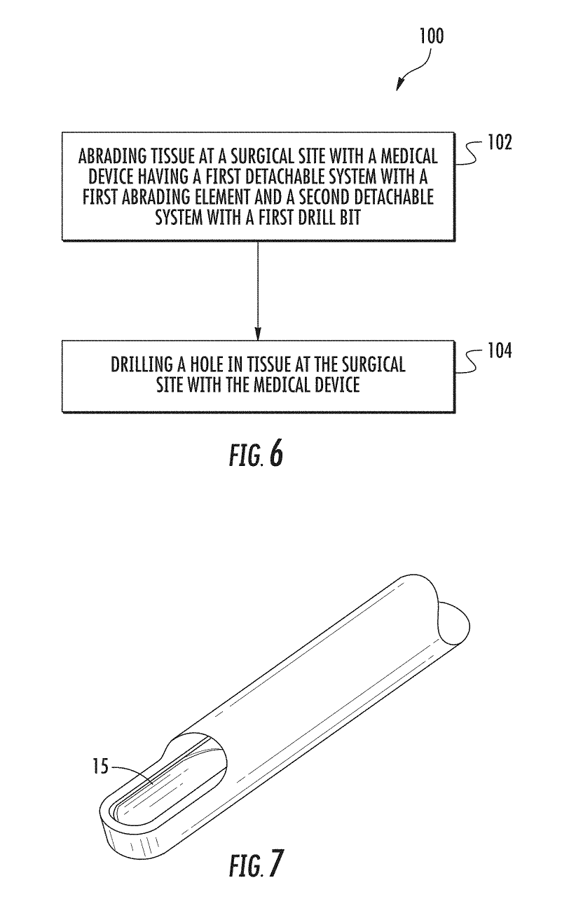

As shown in FIG. 6, a method 100 of preparing a surgical site 30 within a patient for affixing at least one fixation device 12 may include abrading tissue at 102 at the surgical site with the medical device 10, whereby the medical device 10 may include a handpiece 18, an inner drive shaft 36 rotatably positioned within the handpiece 18, a receiver hub 40 formed at a distal end 42 of the handpiece 18 and the inner drive shaft 36, and a first detachable system 20. The first detachable system 20 may include an outer housing 44, an inner drive shaft 36 rotatably positioned within the outer housing 44, a first abrading element 14 attached to a distal end 42 of the inner drive shaft 36 and at least partially extending from the outer housing 44, whereby the first abrading element 14 may be configured to be used to abrade tissue. The first detachable hub 22 may be attached to the outer housing 44 and configured to couple the first detachable hub 22 to the receiver hub 40 such that the inner drive shaft 46 of the first detachable system 20 is placed in mechanical communication with the inner drive shaft 36 positioned within the handpiece 18. The medical device 10 may include a second detachable system 24 including a first drill bit 54 extending distally from a second detachable hub 26, whereby the first drill bit 54 may be configured to be used to drill a hole 32 in tissue 28. The second detachable system 24 may include a second detachable hub 26 configured to couple the second detachable hub 26 to the receiver hub 40 such that the second detachable system 24 is placed in mechanical communication with the inner drive shaft 36 positioned within the handpiece 18. The step of abrading tissue 32 at 102 at the surgical site 30 with the medical device 10 may include abrading tissue 32 at 102 at the surgical site 30 with the medical device 10 with a first detachable system 20 having the first abrading element 14 configured as a burr.

The method 100 may also include drilling a hole 32 at 104 in tissue 28 at the surgical site 30 with the medical device 10, whereby the second detachable system 24 is attached to the handpiece 18 such that the second detachable hub 26 is coupled with the receiver hub 40 on the handpiece 18 and the first detachable system 20 is detached from the receiver hub 40 on the handpiece 18. The step of drilling the hole 32 in tissue 28 at 104 at the surgical site 30 with the medical device 10 may include drilling the hole 32 in tissue 28 at 104 at the surgical site 30 with the medical device 10 with the second detachable system 24 including the first drill bit 54 formed from an elongated shaft 66 with at least one flute 68 extending proximally from a cutting edge 70 at a distal end 72 of the elongated shaft 66. The step of drilling a hole 32 at 104 in tissue 28 at the surgical site 30 with the medical device 10 may include drilling the hole 32 in tissue 28 at the surgical site 30 with the medical device 10 with the second detachable system 24 including the first drill bit 54 having a cutting edge 70 configured to create a hole 32 with a first diameter. The step of drilling a hole 32 at 104 in tissue 28 at the surgical site 30 with the medical device 10 may include drilling the hole 32 in tissue at the surgical site 30 with the medical device 10 with a third detachable system 78. The third detachable system 78 may include a second drill bit 84 extending distally from the third detachable hub 80. The second drill bit 84 may be configured to be used to drill a hole 32 in tissue 28, whereby the hole 32 has a second diameter that differs from the first diameter of a hole 32 formed by the first drill bit 54. The step of drilling a hole 32 at 104 in tissue 28 at the surgical site 30 with the medical device 10 may include drilling the hole 32 in tissue at the surgical site 30 with the medical device 10 with either the second detachable system 24 having the first drill bit 54 configured to create a hole or the third detachable system 78 having the second drill bit 84 configured to create a hole with a diameter different than a diameter of a hole created by the first drill bit 54. The different sized drill bits 54, 84 provide additional flexibility to a user of the device 10.

The foregoing is provided for purposes of illustrating, explaining, and describing embodiments of this invention. Modifications and adaptations to these embodiments will be apparent to those skilled in the art and may be made without departing from the scope or spirit of this invention.

We claim:

1. A medical device with an interchangeable head for abrading tissue, comprising:
a handpiece;
an inner drive shaft rotatably positioned within the handpiece;
a receiver hub formed at a distal end of the handpiece and the inner drive shaft;
a first detachable system, comprising:
an outer housing;
an inner drive shaft rotatably positioned within the outer housing;
a first abrading element attached to a distal end of the inner drive shaft
and at least partially extending from the outer housing, wherein the first abrading element is configured to be used to abrade tissue; and
a first detachable hub attached to the outer housing and configured to couple the first detachable hub to the receiver hub such that the inner drive shaft of the first detachable system is placed in mechanical communication with the inner drive shaft positioned within the handpiece;
a second detachable system, comprising:
a first drill bit configured to be used to drill a hole in tissue;
a second detachable hub configured to couple the second detachable hub to the receiver hub such that the first drill bit of the second detachable system is placed in mechanical communication with the inner drive shaft positioned within the handpiece; and
wherein the first drill bit extends distally from the second detachable hub.

2. The medical device of claim 1, wherein the first abrading element is a burr.

3. The medical device of claim 1, wherein the first abrading element is a shaver.

4. The medical device of claim 1, wherein the first drill bit is formed from an elongated shaft with at least one flute extending proximally from a cutting edge at a distal end of the elongated shaft.

5. The medical device of claim 4, wherein the first drill bit has a cutting edge configured to create a hole with a first diameter.

6. The medical device of claim 1, wherein the first drill bit is formed from a step drill bit.

7. The medical device of claim 1, further comprising,
a third detachable system, comprising:
a second drill bit configured to be used to drill a hole in tissue;
a third detachable hub configured to couple the third detachable hub to the receiver hub such that the second drill bit of the third detachable system is placed in mechanical communication with the inner drive shaft positioned within the handpiece; and
wherein the second drill bit extends distally from the second detachable hub.

8. The medical device of claim 7, wherein a diameter of the first drill bit differs from a diameter of the second drill bit.

9. A medical device with an interchangeable head for abrading tissue, comprising:
a handpiece;
an inner drive shaft rotatably positioned within the handpiece;
a receiver hub formed at a distal end of the handpiece and the inner drive shaft;
a first detachable system, comprising:
an outer housing;
an inner drive shaft rotatably positioned within the outer housing;
a first abrading element attached to a distal end of the inner drive shaft
and at least partially extending from the outer housing, wherein the first abrading element is configured to be used to abrade tissue; and
a first detachable hub attached to the outer housing and configured to couple the first detachable hub to the receiver hub such that the inner drive shaft of the first detachable system is placed in mechanical communication with the inner drive shaft positioned within the handpiece;
a second detachable system, comprising:
a first drill bit configured to be used to drill a hole in tissue;
a second detachable hub configured to couple the second detachable hub to the receiver hub such that the first drill bit of the second detachable system is placed in mechanical communication with the inner drive shaft positioned within the handpiece;
wherein the first drill bit extends distally from the second detachable hub;
wherein the first drill bit has a cutting edge configured to create a hole with a first diameter; and
a third detachable system, comprising:
a second drill bit configured to be used to drill a hole in tissue, whereby the hole has a second diameter that differs from the first diameter of a hole formed by the first drill bit;
a third detachable hub configured to couple the thrid detachable hub to the receiver hub such that the second drill bit of the third detachable system is placed in mechanical communication with the inner drive shaft positioned within the handpiece; and
wherein the second drill bit extends distally from the second detachable hub.

10. The medical device of claim 9, wherein the first abrading element is a burr.

11. The medical device of claim 9, wherein the first abrading element is a shaver.

12. The medical device of claim 9, wherein the first drill bit is formed from an elongated shaft with at least one flute extending proximally from a cutting edge at a distal end of the elongated shaft.

13. The medical device of claim 9, wherein a diameter of the first drill bit differs from a diameter of the second drill bit.

14. The medical device of claim 9, wherein the first drill bit is formed from a step drill bit.

15. A method of preparing a surgical site within a patient for affixing at least one fixation device, comprising:
abrading tissue at the surgical site with a medical device, the medical device comprising:
a handpiece;
an inner drive shaft rotatably positioned within the handpiece;
a receiver hub formed at a distal end of the handpiece and the inner drive shaft;
a first detachable system, comprising:
an outer housing;
an inner drive shaft rotatably positioned within the outer housing;
a first abrading element attached to a distal end of the inner drive shaft and at least partially extending from the outer housing, wherein the first abrading element is configured to be used to abrade tissue; and
a first detachable hub attached to the outer housing and coupled to the first detachable hub to the receiver hub such that the inner drive shaft of the first detachable system is placed in mechanical communication with the inner drive shaft positioned within the handpiece; and
a second detachable system, comprising:
a first drill bit configured to be used to drill a hole in tissue;
a second detachable hub configured to couple the second detachable hub to the receiver hub such that the first drill bit of the second detachable system is placed in mechanical communication with the inner drive shaft positioned within the handpiece; and
wherein the first drill bit extends distally from the second detachable hub; and
drilling a hole in tissue at the surgical site with the medical device, whereby the second detachable system is attached to the handpiece such that the second detachable hub is coupled with the receiver hub on the handpiece and the first detachable system is detached from the receiver hub on the handpiece.

16. The method of claim 15, wherein abrading tissue at the surgical site with a medical device comprises abrading tissue at the surgical site with a medical device with a first detachable system having the first abrading element configured as a burr.

17. The method of claim 15, wherein abrading tissue at the surgical site with a medical device comprises abrading tissue at the surgical site with a medical device with a first detachable system having the first abrading element configured as a shaver.

18. The method of claim 15, wherein drilling the hole in tissue at the surgical site with the medical device comprises drilling the hole in tissue at the surgical site with the medical device with the second detachable system including the first drill bit formed from an elongated shaft with at least one flute extending proximally from a cutting edge at a distal end of the elongated shaft.

19. The method of claim 18, wherein drilling the hole in tissue at the surgical site with the medical device comprises drilling the hole in tissue at the surgical site with the medical device with the second detachable system including the first drill bit having a cutting edge configured to create a hole with a first diameter.

20. The method of claim 15, wherein drilling the hole in tissue at the surgical site with the medical device comprises drilling the hole in tissue at the surgical site with the medical device with a third detachable system, comprising:
a second drill bit configured to be used to drill a hole in tissue;
a third detachable hub configured to couple the thrid detachable hub to the receiver hub such that the second drill bit of the third detachable system is placed in mechanical communication with the inner drive shaft positioned within the handpiece;
wherein the second drill bit extends distally from the second detachable hub; and
wherein the second drill bit is configured to create a hole with a diameter different than a diameter of a hole created with the first drill bit.
